SEQUENCE LISTING (12 sequences)

SEQ ID NO: 1 - DNA, 30 bp, Artificial Sequence
  VP1 forward primer F1 for PCR
gcggaattcatggcaagacgagctcgcaga

SEQ ID NO: 2 - DNA, 30 bp, Artificial Sequence
  VP1 reverse primer R1 for PCR
gggctcgagtcagggctgcgtcccccagta

SEQ ID NO: 3 - DNA, 1350 bp, Chicken anemia virus
atggcaagacgagctcgcagaccgagaggccgattttacgccttcagaagaggacggtggcacaacctcaagcgacttcgacgaagatataaatttcgacatcggaggagacagcggtatcgtagacgagcttttaggaaggcctttcacaacccccgccccggtacgtatagtgtgaggctgccgaacccgcaatctactatgactatccgcttccaaggaatcatatttctcacggaaggactcattctgcctaaaaacagcacagcggggggctatgcagaccacttgtacggggcgagagtcgccaagatctccgtgaacctgaaagagttcctgctagcgtcaatgaacctgacatacgtgggcaaaatcggaggccccatcgccggtgagttgattgcggacgggtctcagtctcaagccgcgcagaactggcctaattgctggctgccgctagataataacgtgccctccgcgacaccatcggcatggtggagatgggccttaatgatgatgcagcccacggactcttgccggttctttaatcaccctaagcagatgaccctgcaagacatgggtcgtatgtttgggggccggcacctgttccgacacattgaaacacgctttcagctccttgccactaagaatgagggatccttcagccccgtggcgagtcttctctcccagggagagtacctcacgcgtcgggacgatgttaaatacagcagcgatcaccagaaccgatggcgaaaaggcgaacagccgatgacggggggtattgcttatgcgaccgggaaaatgagacccgacgagcaacagtaccctgctatgcccccagaccccccgataatcaccgctactacagcgcaaggaacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaaaggccagagatggcacacgctggtgccgctgggaacggagaccatcaccgacagctacatgagtgcacccgcatcagagctggacactaatttctttacgctttacgtagcgcaaggcacaaataagtcgcagcagtacaagttcggcacagctacatacgcgctaaaggagccagtaatgaagagcgatgcatgggcagtggtacgcgtccagtcggtctggcaacttggtaacaggcagaggccatacccatgggacgtaaactgggccaacagcaccatgtactgggggacgcagccctga

SEQ ID NO: 4 - DNA, 321 bp, Artificial Sequence
  5′ end codon-optimized vp1 gene
atggctcgtcgtgctcgtcgtccgcgtggtcgtttctacgctttccgtcgtggtcgttggcacaacctgaaacgtctgcgtcgtcgttacaaattccgtcaccgtcgtcgtcagcgttaccgtcgtcgtgctttccgtaaagctttccacaacccgcgtccgggtacctactctgttcgtctgccgaacccgcagtctaccatgaccatccgtttccagggtatcatcttcctgaccgaaggtctgatcctgccgaaaaactctaccgctggtggttacgctgaccacctgtacggtgctcgtgttgctaaaatctctgtt

SEQ ID NO: 5 - DNA, 3255 bp, Artificial Sequence
  recombinant vector pBH-co-N
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacggccgtcaaggccaagcttcccgaattcatggctcgtcgtgctcgtcgtccgcgtggtcgtttctacgctttccgtcgtggtcgttggcacaacctgaaacgtctgcgtcgtcgttacaaattccgtcaccgtcgtcgtcagcgttaccgtcgtcgtgctttccgtaaagctttccacaacccgcgtccgggtacctactctgttcgtctgccgaacccgcagtctaccatgaccatccgtttccagggtatcatcttcctgaccgaaggtctgatcctgccgaaaaactctaccgctggtggttacgctgaccacctgtacggtgctcgtgttgctaaaatctctgttctcgaggggatatcacgtgaagcttgcggccgcacatgggccaagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 6 - DNA, 30 bp, Artificial Sequence
  opt-VP1 forward primer F for PCR
cccgaattcatggctcgtcgtgctcgtcgt

SEQ ID NO: 7 - DNA, 48 bp, Artificial Sequence
  opt-VP1 reverse primer R for PCR
cgctagcaggaactctttcaggttaacagagattttagcaacacgagc

SEQ ID NO: 8 - DNA, 24 bp, Artificial Sequence
  VP1 forward primer F2 for PCR
aacctgaaagagttcctgctagcg

SEQ ID NO: 9 - DNA, 1029 bp, Artificial Sequence
  partial vp1 gene
aacctgaaagagttcctgctagcgtcaatgaacctgacatacgtgggcaaaatcggaggccccatcgccggtgagttgattgcggacgggtctcagtctcaagccgcgcagaactggcctaattgctggctgccgctagataataacgtgccctccgcgacaccatcggcatggtggagatgggccttaatgatgatgcagcccacggactcttgccggttctttaatcaccctaagcagatgaccctgcaagacatgggtcgtatgtttgggggccggcacctgttccgacacattgaaacacgctttcagctccttgccactaagaatgagggatccttcagccccgtggcgagtcttctctcccagggagagtacctcacgcgtcgggacgatgttaaatacagcagcgatcaccagaaccgatggcgaaaaggcgaacagccgatgacggggggtattgcttatgcgaccgggaaaatgagacccgacgagcaacagtaccctgctatgcccccagaccccccgataatcaccgctactacagcgcaaggaacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaaaggccagagatggcacacgctggtgccgctgggaacggagaccatcaccgacagctacatgagtgcacccgcatcagagctggacactaatttctttacgctttacgtagcgcaaggcacaaataagtcgcagcagtacaagttcggcacagctacatacgcgctaaaggagccagtaatgaagagcgatgcatgggcagtggtacgcgtccagtcggtctggcaacttggtaacaggcagaggccatacccatgggacgtaaactgggccaacagcaccatgtactgggggacgcagccctga

SEQ ID NO: 10 - DNA, 1350 bp, Artificial Sequence
  codon-optimized full length vp1 gene
atggctcgtcgtgctcgtcgtccgcgtggtcgtttctacgctttccgtcgtggtcgttggcacaacctgaaacgtctgcgtcgtcgttacaaattccgtcaccgtcgtcgtcagcgttaccgtcgtcgtgctttccgtaaagctttccacaacccgcgtccgggtacctactctgttcgtctgccgaacccgcagtctaccatgaccatccgtttccagggtatcatcttcctgaccgaaggtctgatcctgccgaaaaactctaccgctggtggttacgctgaccacctgtacggtgctcgtgttgctaaaatctctgttaacctgaaagagttcctgctagcgtcaatgaacctgacatacgtgggcaaaatcggaggccccatcgccggtgagttgattgcggacgggtctcagtctcaagccgcgcagaactggcctaattgctggctgccgctagataataacgtgccctccgcgacaccatcggcatggtggagatgggccttaatgatgatgcagcccacggactcttgccggttctttaatcaccctaagcagatgaccctgcaagacatgggtcgtatgtttgggggccggcacctgttccgacacattgaaacacgctttcagctccttgccactaagaatgagggatccttcagccccgtggcgagtcttctctcccagggagagtacctcacgcgtcgggacgatgttaaatacagcagcgatcaccagaaccgatggcgaaaaggcgaacagccgatgacggggggtattgcttatgcgaccgggaaaatgagacccgacgagcaacagtaccctgctatgcccccagaccccccgataatcaccgctactacagcgcaaggaacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaaaggccagagatggcacacgctggtgccgctgggaacggagaccatcaccgacagctacatgagtgcacccgcatcagagctggacactaatttctttacgctttacgtagcgcaaggcacaaataagtcgcagcagtacaagttcggcacagctacatacgcgctaaaggagccagtaatgaagagcgatgcatgggcagtggtacgcgtccagtcggtctggcaacttggtaacaggcagaggccatacccatgggacgtaaactgggccaacagcaccatgtactgggggacgcagccctga

SEQ ID NO: 11 - protein, 449 aa, Chicken anemia virus
MARRARRPRGRFYAFRRGRWHNLKRLRRRYKFRHRRRQRYRRRAFRKAFHNPRPGTYSVRLPNPQSTMTIRFQGIIFLTEGLILPKNSTAGGYADHLYGARVAKISVNLKEFLLASMNLTYVGKIGGPIAGELIADGSQSQAAQNWPNCWLPLDNNVPSATPSAWWRWALMMMQPTDSCRFFNHPKQMTLQDMGRMFGGRHLFRHIETRFQLLATKNEGSFSPVASLLSQGEYLTRRDDVKYSSDHQNRWRKGEQPMTGGIAYATGKMRPDEQQYPAMPPDPPIITATTAQGTQVRCMNSTQAWWSWDTYMSFATLTALGAQWSFPPGQRSVSRRSFNHHKARGAGDPKGQRWHTLVPLGTETITDSYMSAPASELDTNFFTLYVAQGTNKSQQYKFGTATYALKEPVMKSDAWAVVRVQSVWQLGNRQRPYPWDVNWANSTMYWGTQP

SEQ ID NO: 12 - protein, 449 aa, Chicken anemia virus
  MISC_FEATURE (14)..(14): Xaa can be Ala or Ser
MARRARRPRGRFYXFRRGRWHXLKRLXRRYKFRHRRRQRYRRRAFRKAFHNPRPGTYSVRLPNPQSTMTIRFQGXIFLTEGLILPKNSTAGGYADHXYGARVAKISVNLKEFLLASMNLTYVGKIGGPIAGELIADGSXSQAAQNWPNCWLPLDNNVPSATPSAWWRWALMMMQPTDSCRFFNHPKQMTLQDMGRMFGGXHLFRHIETRFQLLATKNEGSFSPVASLLSQGEYLTRRDDVKYSSDHQNRWXKGXQPMTGGIAYATGKMRPDEQQYPAMPPDPPIITATTAQGTQVRCMNSTQAWWSWDTYMSFATLTALGAQWSFPPGQRSVSRRSFNHHKARGAGDPKGQRWHTLVPLGTETITDSYMSAPASELDTNFFTLYVAQGTNKSQQYKFGTATYALKEPVMKSDAWAVVRVQSVWQLGNRQRPYPWDVNWANSTMYWGTQP

(19) United States
(12) Patent Application Publication
(10) Pub. No.: US 2013/0023036 A1
(43) Pub. Date: Jan. 24, 2013

(54) NUCLEIC ACID CONSTRUCT, RECOMBINANT VECTOR, AND RECOMBINANT E. COLI PRODUCING CHICKEN ANEMIA VIRUS VP1 PROTEIN

(71) Applicants: Meng-Shiou Lee, Taichung City (TW); Guan-Hua Lai, Taichung City (TW); Yi-Yang Lien, Taichung City (TW)

(72) Inventors: Meng-Shiou Lee, Taichung City (TW); Guan-Hua Lai, Taichung City (TW); Yi-Yang Lien, Taichung City (TW)

(73) Assignee: CHINA MEDICAL UNIVERSITY, Taichung City (TW)

(21) Appl. No.: 13/547,016

(22) Filed: Jul. 11, 2012

Related U.S. Application Data
(60) Provisional application No. 61/506,851, filed on Jul. 12, 2011.

(30) Foreign Application Priority Data
Jul. 14, 2011 (TW) 100124934
Mar. 7, 2012 (TW) 101107705

(51) Int. Cl. C12N 15/70; C12N 1/21

(52) U.S. Cl. 435/252.33; 435/320.1

(57) ABSTRACT

Disclosed herein is an expression cassette adapted to be expressed in an E. coli host cell and having a first nucleic acid fragment encoding a full-length chicken anemia virus (CAV) VP1 protein. In particular, the first nucleic acid fragment has a 5′-region that encodes a N-terminal amino acid sequence of the full-length CAV VP1 protein and is codon-optimized as compared to a corresponding 5′-region of a wild-type CAV vp1 gene, thus to encode the full-length VP1 protein. Specifically, the optimized codons are introduced into the corresponding 5′-region of the wild-type CAV vp1 gene by codon replacements.

### CROSS-REFERENCE TO RELATED APPLICATION

This application claims priority of U.S. Provisional Application No. 61/506,851, filed on Jul. 12, 2011, and priorities of Taiwanese Application Nos. 100124934 and 101107705, filed on Jul. 14, 2011 and Mar. 7, 2012, respectively.

### BACKGROUND OF THE INVENTION

1. Field of the Invention

This invention relates to an expression cassette adapted to be expressed in an E. coli host cell and comprising a first nucleic acid fragment encoding a full-length chicken anemia virus (CAV) VP1 protein. In particular, the first nucleic acid fragment has a 5′-region that encodes a N-terminal amino acid sequence of the full-length CAV VP1 protein and is codon-optimized to encode the full-length CAV VP1 protein. The 5′-region of the first nucleic acid fragment contains therein optimized codons that are introduced into the corresponding 5′-region of the wild-type CAV vp1 gene by codon optimization that includes rare codon replacements.

2. Description of the Related Art

Chicken anemia virus (CAV), also known as chicken infectious anemia virus (CIAV), is a non-enveloped circular single-stranded DNA virus that leads to a severe immunosuppressive syndrome and anemia in chickens.

There are three open reading frames (ORFs) in the CAV genome, which encode three virus proteins, i.e., VP1, VP2 and VP3. VP1 is a 51 kDa structural protein, which is responsible for assembly of viral capsid. VP2 is a 24 kDa non-structural protein with dual specificity phosphatase activity, which is responsible for infection, assembly and replication. Studies have shown that VP1 and VP2 allow the elicitation of host-produced virus neutralizing antibodies (Koch G. et al. (1995), Vaccine, 13:763-770). Finally, VP3 is a 13 kDa protein, which is a well-known apoptin that can induce apoptosis in cells of the infected chickens.

It has been disclosed that the complete coding sequence for VP1 protein in a Taiwanese wild isolate of CAV (i.e., CIA-89 isolate) exhibits 96.4% and 97.1% sequence identity to that of USA isolate L-028 (NCBI GenBank Assession No. U69549 and AAC55986 for nucleotide and amino acid sequences of L-028, respectively) and that of German isolate Cuxhaven-1 (NCBI GenBank Assession No. M55918, base pairs 853 to 2202, and NCBI GenBank Assession No. AAA91824 for amino acid sequence) (Chen-Ru Yang; “Study of Molecular Cloning and Expression of the VP1 and VP2 Genes in Chicken Infectious Anemia Virus”; National Pingtung University of Science and Technology Master of Science thesis; 2004). Thus, VP1 protein is thought to be a good candidate to act as an immunogen when developing subunit vaccines and diagnostic kits.

Up to the present, Escherichia coli (E. coli) is a widely accepted host cell used to express VP1 protein. For example, Pallister J. et al. have attempted to develop an indirect enzyme-linked immunosorbent assay (ELISA) using glutathione-S-transferase (GST) tagged VP1 fusion protein expressed by E. coli for the detection of anti-CAV antibody in a CAV-infected chicken serum (Pallister J. et al. (1994), Vet. Microbiol., 39:167-178). Results have shown that a fusion protein containing GST and full length VP1 was broken down to a product of 30-34 kDa. Conversely, a GST fusion protein with truncated VP1 protein that was designed to eliminate first 67 amino acids showed little apparent breakdown. It has been postulated that this breakdown was attributed to a series of arginine residues at the N-terminal region of the VP1 protein. This highly positive-charged region may act as a DNA binding protein, as it shows homology to other DNA binding histone proteins, and is likely to be rapidly degraded.

It was disclosed by the present inventors that a serial N-terminus deletions of the VP1 protein, i.e., the first 30, 60 and 129 amino acids were truncated from the VP1 protein, were created in order to evaluate VP1 protein expression (Lee M. S. et al. (2009), Process Biochem., 44:390-395). The results demonstrated that all three of these truncated VP1 proteins can be expressed in E. coli, in which the VP1 protein with the deletion of 129 amino acid residues at N-terminus region exhibited the highest expression level compared to the other two proteins. However, this deletion leads to a lower antigenicity with anti-CAV antibodies, possibly due to the loss of antigenic sites at the N-terminus of the VP1 protein.

E. coli remains a popular host for the expression of heterologous proteins, in which the expression of the heterologous protein is dependent on the codons thereof. Rare codons for E. coli includes agg and aga for arginine, ccc for proline, and ggg for glycine, etc. (Kane J. F. (1995), Curr. Opin. Biotechnol., 6:494-500). In order to highly express heterologous proteins in E. coli, a popular approach is to substitute rare codons to codons that are in favor of E. coli by codon optimization (Bouallag N. et al. (2009), Protein Expr. Purif., 67:35-40).

Despite many studies reporting successes of codon optimization, failure in codon optimization still exists, which might potentially be due to reasons related to “over-optimization” including (1) imbalanced tRNA pool caused by strongly transcribed mRNAs that leads to translational error; (2) inhibition of ribosome processivity due to repetitive elements and secondary structures in the gene and mRNA introduced during codon optimization; and (iii) elimination of non-optimal codons which are important for folding of nascent translated polypeptide (Chuan Y. P. et al. (2008), J Biotechnol., 134:64-71).

From the above, it is known that, production of the recombinant full-length VP1 protein has generally been unsuccessful because of a span of amino acids at the N-terminus of the VP1 protein that is highly rich in arginine residue. Furthermore, VP1 has been proposed to be cytotoxic in an E. coli expression system. Once the N-terminus of VP1 is deleted, protein expression is improved significantly. Nevertheless, the N-terminus of VP1 may still be involved in eliciting neutralizing antibodies because it contains some functional epitopes. Thus, there is a need to overcome the difficulties that have been encountered during the production of full-length VP1 protein using the E. coli expression system.

### SUMMARY OF THE INVENTION

Therefore, the present invention provides an expression cassette adapted to be expressed in an E. coli host cell and comprising a first nucleic acid fragment encoding a full-length chicken anemia virus (CAV) VP1 protein. The first nucleic acid fragment has a 5′-region that encodes a N-terminal amino acid sequence of the full-length CAV VP1 protein and is codon-optimized as compared to a corresponding 5′-region of a wild-type CAV vp1 gene encoding the full-length VP1 protein. The 5′-region of the first nucleic acid fragment contains therein optimized codons that are introduced into the corresponding 5′-region of the wild-type CAV vp1 gene by codon optimization that includes the following rare codon replacements:
replacing a glycine codon of gga, ggc or ggg with a codon of ggt;
replacing a leucine codon of ctc, ctt or ttg with a codon of ctg;
replacing a threonine codon of aca, act or acg with a codon of acc; replacing an isoleucine codon of ata or att with a codon of atc;
replacing a glutamine codon of caa with a codon of cag;
replacing an arginine codon of aga, agg, cga, cgc or cgg with a codon of cgt; and
replacing a proline codon of ccc or cct with a codon of ccg.

### BRIEF DESCRIPTION OF THE DRAWINGS

Other features and advantages of the present invention will become apparent in the following detailed description of the preferred embodiment of the invention, with reference to the accompanying drawings, in which:

FIG. 1 shows the construct of an expression vector pET-28a, in which Kan^{r} represents a kanamycin resistance gene; XhoI and EcoRI represent the restriction sites for the corresponding restriction enzymes; 6×His represents hexahistidine tag;

FIG. 2 shows the construct of an expression vector pGEX-4T-1; Amp^{r} represents an ampicillin resistance gene; XhoI and EcoRI represent the restriction sites for the corresponding restriction enzymes; GST represents glutathione S-transferase tag;

FIG. 3 shows the construct of a recombinant plasmid pET-VP1, in which Kan^{r} represents a kanamycin resistance gene; vp1 represents a gene that encodes a VP1 protein of the CAV Taiwan CIA-89 strain; XhoI and EcoRI represent the restriction sites for the corresponding restriction enzymes; 6×His represents hexahistidine tag;

FIG. 4 shows the construct of a recombinant plasmid pGEX-VP1; Amp^{r} represents an ampicillin resistance gene; vp1 represents a gene that encodes a VP1 protein of the CAV Taiwan CIA-89 strain; XhoI and EcoRI represent the restriction sites for the corresponding restriction enzymes; GST represents glutathione S-transferase tag;

FIG. 5 shows Western blots of VP1 protein expression in E. coli BL21 (DE3) using various expression vectors and artificial constructs; the upper panel shows VP1 protein expression by artificial constructs pET-VP1 and pGEX-VP1 that were detected with anti-His tag and anti-GST tag monoclonal antibodies respectively; the lower panels show the respective expression vectors used in the panel directly above; the solid triangle indicates a clear band of VP1 protein recognized by monoclonal anti-GST antibody at approximately 78 kDa; the hollow triangle indicates the GST tag;

FIG. 6 is a schematic flow chart of obtaining a full length optimized DNA fragment of vp1 gene by PCR overlapping strategy, in which the opt-vp1 DNA fragment contains a codon-optimized 5′ end of the vp1 gene fused with the 3′ end of the wild-type vp1 gene;

FIG. 7 shows a construct of a recombinant plasmid pGEX-opt-VP1, Amp^{r} represents an ampicillin resistance gene; codon optimized full length vp1 gene represent a vp1 gene of SEQ ID NO: 10; XhoI and EcoRI represents the restriction sites for the corresponding restriction enzymes; GST represents glutathione S-transferase tag;

FIG. 8 shows protein levels of GST-VP1 and GST-opt-VP1 fusion proteins expressed by E. coli containing recombinant plasmid, pGEX-VP1, and E. coli containing recombinant plasmid, pGEX-opt-VP1, before and after IPTG induction; and

FIG. 9 is a plot showing the optical density absorbance (OD₄₀₅) using indirect enzyme linked immunosorbent assay (ELISA), demonstrating the antigenicity of the recombinant GST-opt-VP1 protein using chicken sera with or without chicken anemia virus infection.

### DETAILED DESCRIPTION OF THE PREFERRED EMBODIMENTS

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which this invention belongs. One skilled in the art will recognize many methods and materials similar or equivalent to those described herein, which could be used in the practice of the present invention. Indeed, the present invention is in no way limited to the methods and materials described.

A DNA “coding sequence” is a double-stranded DNA sequence which is transcribed into RNA, and the RNA is translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, sequences from the genomes of viruses that infect prokaryotes or eukaryotes, cDNA from eukaryotic mRNA, genomic DNA sequences from eukaryotic (e.g., mammalian) DNA, and even synthetic DNA sequences. A “cDNA” is defined as copy-DNA or complementary-DNA, and is a product of a reverse transcription reaction from a mRNA transcript.

The terms “nucleic acid”, “nucleic acid sequence” and “nucleic acid fragment” as used herein refer to a deoxyribonucleotide or ribonucleotide sequence in single-stranded or double-stranded form, that comprises naturally occurring and known nucleotides or artificial chemical mimics. The term “nucleic acid” as used herein is interchangeable with the terms “gene,” “cDNA,” “mRNA,” “oligo-nucleotide” and “polynucleotide” in use.

As used herein, the term “DNA fragment” refers to a DNA polymer, in the form of a separate segment or as a component of a larger DNA construct, which has been derived either from isolated DNA or synthesized chemically or enzymatically such as by methods disclosed elsewhere.

As used herein, the term “fusion gene” refers to a DNA fragment in which two or more genes are fused in a single reading frame to encode two or more proteins that are fused together via one or more peptide bonds. As used herein, the term “fusion protein” refers to a protein or polypeptide encoded by a fusion gene and it may be used interchangeably with the term “fusion gene product.”

The term “expression cassette” refers to a nucleic acid molecule capable of directing expression of a particular nucleotide sequence in an appropriate host cell, including a promoter operably linked to the nucleotide of interest, termination signals and one or more restriction enzyme sites allowing insertion of heterologous gene sequences. It can also include sequence required for proper translation of the nucleic acid fragment. The expression cassette comprising the nucleic acid fragment of interest can be chimeric, i.e., at least one of its components is heterologous with respect to at least one of its other components.

As used herein, the term “nucleic acid fragment” indicates a polynucleotide molecule that has been isolated or purified and ready to be genetically engineered in protein production systems. The nucleic acid fragment can be obtained by chemical synthesis, recombinant DNA technology or by techniques that are well known to those skilled in the art (such as shuffling experiments, site-directed mutagenesis experiments).

Unless otherwise indicated, the nucleic acid fragment as disclosed by the current invention also implicitly encompasses complementary sequence, conservative analogs, related naturally occurring structural variants and/or synthetic non-naturally occurring analogs thereof. Examples include degenerative codon substitution, deletion, insertion, substitution or addition of the homologous sequences. Specifically, degenerative codon substitution can be achieved by substituting a nucleotide residue at the third position of one or more selected codons in a nucleic acid fragment with other nucleotide residue (s).

As used herein, the term “transcription direction” refers to the direction of 5′ to 3′ addition of nascent RNA transcripts.

As used herein, the term “coding region” refers to a nucleic acid fragment encoding an amino acid that is found in a nascent polypeptide translated from a mRNA molecule.

As used herein, the term “promoter” can be used interchangeably with the term “promoter sequence” and refers to a DNA regulatory region capable of binding RNA polymerase in a cell and initiating transcription of a downstream (3′ direction) coding sequence. The promoter is bound at its 3′ terminus by the translation start codon of a coding sequence and extends upstream (5′ direction) to include a minimum number of bases or elements necessary to initiate transcription. Promoters which cause a gene to be expressed inmost cell types at most times are commonly referred to as “constitutive promoters”. Promoters which cause conditional expression of a structural nucleotide sequence under the influence of changing environmental conditions or developmental conditions are commonly referred to as “inducible promoter.”

The term “operatively connected” as used herein means that a first sequence is disposed sufficiently close to a second sequence such that the first sequence can influence the second sequence or regions under the control of the second sequence. For instance, a promoter sequence may be operatively connected to a gene sequence, and is normally located at the 5′-terminus of the gene sequence such that the expression of the gene sequence is under the control of the promoter sequence. In addition, a regulatory sequence may be operatively connected to a promoter sequence so as to enhance the ability of the promoter sequence in promoting transcription. In such case, the regulatory sequence is generally located at the 5′-terminus of the promoter sequence.

As used herein, the term “upstream” and “downstream” refer to the position of an element of nucleic acid fragment. “Upstream” signifies an element that is more 5′ than the reference element. “Downstream” signifies an element that is more 3′ than the reference element.

The terms “recombinant vector” and “expression vector” as used herein refer to any recombinant expression system capable of expressing a selected nucleic acid fragment, in any host cell in vitro or in vivo, constitutively or inducibly. The expression vector may be an expression system in linear or circular form, and covers expression systems that remain episomal or that integrate into the host cell genome. The expression system may or may not have the ability to self-replicate, and it may drive only transient expression in a host cell.

According to this invention, the term “transformation” can be used interchangeably with the term “transfection” when such term is used to refer to the introduction of an exogenous nucleic acid molecule into a selected host cell. According to techniques known in the art, a nucleic acid molecule (e.g., a recombinant DNA construct or a recombinant vector) can be introduced into a selected host cell by various techniques, such as calcium phosphate- or calcium chloride-mediated transfection, electroporation, microinjection, particle bombardment, liposome-mediated transfection, transfection using bacterial bacteriaphages, transduction using retroviruses or other viruses (such as vaccinia virus or baculovirus of insect cells), protoplast fusion, Agrobacterium-mediated transformation, or other methods.

The terms “cell,” “host cell,” “transformed host cell” and “recombinant host cell” as used herein can be interchangeably used, and not only refer to specific individual cells but also include sub-cultured offsprings or potential offsprings thereof. Sub-cultured offsprings formed in subsequent generations may include specific genetic modifications due to mutation or environmental influences and, therefore, may factually not be fully identical to the parent cells from which the sub-cultured offsprings were derived. However, sub-cultured cells still fall within the coverage of the terms used herein.

The amino acid notations used throughout the specification can be presented in its full name, single- or three-letter abbreviations. In addition, N-terminus refers to the first amino acid present in a peptide and written on the left hand side of an amino acid sequence in a conventional manner. C-terminus refers to the end of the amino acid sequence and is on the right hand side of the sequence. The peptide sequence is written from N- to C-terminus.

The terms “polypeptide,” “peptide” and “protein” as used herein can be interchangeably used, and refer to a polymer formed of amino acid residues, wherein one or more amino acid residues are naturally occurring amino acids or artificial chemical mimics. The term “recombinant polypeptide” or “recombinant protein” as used herein refers to polypeptides or proteins produced by recombinant DNA techniques, i.e., produced from cells transformed by an exogenous DNA construct encoding the desired polypeptide or the desired protein.

VP1 protein is thought to be a good candidate for use as an immunogen when developing subunit vaccines and diagnostic kits. Up to the present, a number of different expression systems have been used to produce VP1 protein, such as genetic-engineered E. coli. However, the applicants are unaware of any patent or publication that discloses a method to successfully express full length VP1 protein in E. coli with high overall yield while maintaining good antigenicity.

Thus, in order to reach significant increase in expression levels of VP1 protein, a full-length chicken anemia virus (CAV) vp1 gene that has a 5′ region that is codon optimized as compared to a corresponding 5-region of wild-type CAV vp1 gene was constructed. Specifically, genomic DNA of CAV, Taiwanese isolate CIA-89 (kindly provided by Professor Yi-Yang Lien of National Pingtung University of Science and Technology) was used as a template to obtain a PCR product of a full length wild-type vp1 gene. A primer pair, VP1 forward primer F1 (SEQ ID NO: 1) and VP1 reverse primer R1 (SEQ ID NO: 2), was designed using the complete coding sequence of the full length wild-type vp1 gene (NCBI GenBank Assession No. U69549). The PCR product having 1,368 bp is represented by SEQ ID NO: 3, which encodes an amino acid sequence of SEQ ID NO: 11.

The full length wild-type vp1 gene from CIA-89 Taiwanese isolate was codon-optimized at the 5′-region from nucleotides 1-321 by codon replacements to obtain an optimized full length vp1 gene as identified by SEQ ID NO: 10. Thereafter, the optimized full length vp1 gene was cloned into an expression vector and transformed into E. coli. The transformed E. coli highly expressed optimized VP1 protein after isopropyl-β-D-thiogalactopyranoside (IPTG) induction as shown in Western blot analyses. The optimized VP1 protein has an amino acid sequence identical to that of Taiwanese isolate CIA-89. The optimized VP1 protein is proven to show good antigenicity as demonstrated by enzyme-linked immunosorbent assay (ELISA).

Thus, the present invention provides an expression cassette adapted to be expressed in an E. coli host cell and comprising a first nucleic acid fragment encoding a full-length chicken anemia virus (CAV) VP1 protein. The first nucleic acid fragment has a 5′-region that encodes a N-terminal amino acid sequence of the full-length CAV VP1 protein and is codon-optimized as compared to a corresponding 5′-region of a wild-type CAV vp1 gene encoding the full-length VP1 protein. The 5′-region of the first nucleic acid fragment contains therein optimized codons that are introduced into the corresponding 5′-region of the wild-type CAV vp1 gene by codon optimization that includes the following rare codon replacements:
replacing a glycine codon of gga, ggc or ggg with a codon of ggt;
replacing a leucine codon of ctc, ctt or ttg with a codon of ctg;
replacing a threonine codon of aca, act or acg with a codon of acc;
replacing an isoleucine codon of ata or att with a codon of atc;
replacing a glutamine codon of caa with a codon of cag;
replacing an arginine codon of aga, agg, cga, cgc or cgg with a codon of cgt; and
replacing a proline codon of ccc or cct with a codon of ccg.

Preferably, the codon optimization further includes the following codon replacements, in which
replacing an alanine codon of gca, gcc or gcg with a codon of gct;
replacing a lysine codon of aag with a codon of cgt;
replacing a histidine codon of cat with a codon of cac;
replacing a phenylalanine codon of ttt with a codon of ttc;
replacing a serine codon of agc, agt or tcc with a codon of tct;

replacing a tyrosine codon of tat with a codon of tac; and
replacing a valine codon of gtc or gtg with a codon of gtt.

The expression cassette adapted to be expressed in an E. coli host cell to encode a full-length chicken anemia virus VP1 protein.

The alignment of the amino acid sequence of full length VP1 proteins from Taiwanese, USA and German isolates showed a sequence homology as represented by a reference sequence, SEQ ID NO: 12, with Xaa representing amino acid residues that differ among the strains.

The term “codon optimization” refers to the process of optimally altering the nucleic acid fragment encoding a protein, polypepetide, antigen, epitope, domain or fragment for expression or translation in various hosts. Specifically, the rare codons in the target gene are substituted with codons that more closely reflect the codon usage of the host cell without modifying the amino acid sequence of the encoded protein. Within the context of the present invention, codon optimization for E. coli was designed according to the information provided by GenScript OptimumGene™ codon optimization software.

Preferably, the N-terminal amino acid sequence of the full-length CAV VP1 protein encoded by the 5′-region of the first nucleic acid fragment has a length of at least 30 to 130 amino acids. One of the preferred embodiments of the present invention is codon optimization of 107 amino acids of the N-terminal amino acid sequence of the full-length CAV VP1 protein encoded by the 5′-region of the first nucleic acid fragment.

Preferably, the expression cassette further comprises a second nucleic acid fragment operably connected to the first nucleic acid fragment and encoding a target protein. Preferably, the target protein is a protein tag, an antibody, an antigen, an antimicrobial peptide, a hormone peptide or an enzyme.

Preferably, the second nucleic acid fragment is located upstream of the first nucleic acid fragment, so that the first and second nucleic acid fragments together encode a fusion protein of the full-length CAV VP1 protein and the target protein, wherein the target protein is located upstream of the N-terminal acid sequence of the full-length CAV VP1 protein.

In this present invention, the target protein is a protein tag that can help the full length chicken anemia virus VP1 protein fold and express in E. coli. Preferably, the target protein is a protein tag selected from the group consisting of glutathione-S-transferase, hexahistidine tag, maltose binding protein, small ubiquitin-like modifier, and combinations thereof. In an embodiment of this invention, the target protein is glutathione-S-transferase.

In this invention, the expression cassette further comprises a promoter that operably controls the expression of nucleic acid fragments. The promoter suitable for use in this invention include, but are not limited to, tac promoter, T3 promoter, T5 promoter, T7 promoter, PBAD promoter, PL promoter, PRHA promoter, tet promoter and lac promoter. Tac promoter is used in one of the preferred embodiments of the present invention.

The expression cassette can be in the form of a recombinant vector.

The recombinant vector which harbors the first nucleic acid fragment as described above can be made using conventional techniques that are well known to a skilled artisan. A vector that is suitable for producing the recombinant vector includes bacteriophages, plasmids, cosmids, viruses or retroviruses. Preferably, the vector can be selected from the group consisting of pGEX-4T-1, pET-28a, pMAL and pET-SUMO. The vector pGEX-4T-1 is used in one of the preferred embodiments of the current invention.

Preferably, the recombinant vector may include other expression control elements, such as a transcription starting site, a transcription termination site, a ribosome binding site, a RNA splicing site, a polyadenylation site, a translation termination site, etc.

Preferably, the recombinant vector may further include regulatory elements, such as transcription/translation enhancer sequences, a Shine-Dalgarno sequence, a regulatory sequence and at least a marker gene (such as antibiotic-resistance gene) or reporter gene allowing for the screening of the recombinant vectors under suitable conditions.

According to this invention, the recombinant vector can be transformed into a desired E. coli strain. The present invention provides a recombinant E. coli harboring the recombinant vector described above.

E. coli strains suitable for the present invention include, but are not limited to, BL21 (DE3)-pLysS, BL21 (DE3)-pLysE, BL21 Star (DE3)-pLysS, Tuner (DE3)-pLysS, Origami B (DE3)-pLysS, Rosetta (DE3)-pLysS, Rosetta-gami (DE3)-pLysS, NovaBlue (DE3), C41 (DE3) and C43 (DE3). BL21 (DE3)-pLysS is used in one of the preferred embodiments of the present invention.

Culture media and culture conditions for host cells suitable for carrying out DNA recombination techniques are well known in the field of biotechnology. For instance, host cells may be cultured in a fermentation bioreactor, a shaking flask, a test tube, a microtiter plate, or a petri dish, and cultivation of the host cells may be conducted under conditions suitable for growth of said cells, including the culture temperature, the pH value of the culture medium, and the dissolved oxygen concentration of the culture.

Preferably, the expression cassette may further include a third nucleic acid fragment encoding CAV VP2 protein. The third nucleic acid fragment can be cloned into the recombinant vector described above or cloned into another plasmid or vector expressible in E. coli host cell to allow concordant expression of VP1 and VP2 proteins.

The invention also provides a method to express a full length CAV VP1 protein, which includes the steps of culturing the recombinant E. coli under suitable conditions and harvesting the full length CAV VP1 protein.

Since the antigenicity of the optimized full length VP1 protein provided by the present invention is well preserved, utilization thereof for use in the development of diagnostic kits or vaccines is desirable.

The optimized CAV VP1 protein from the present invention can be used to detect CAV antibodies in CAV-infected chicken sera using diagnostic assay kits. Examples of such kits include, but are not limited to, enzyme linked immunosorbent assay (ELISA), fluorescent immunoassay (FIA), chemiluminescent immunoassay and Western blotting. VP1 protein can also be used together with VP2 protein for the elicitation of neutral antibodies for the chicken.

### EXAMPLES

### <Materials and Methods>

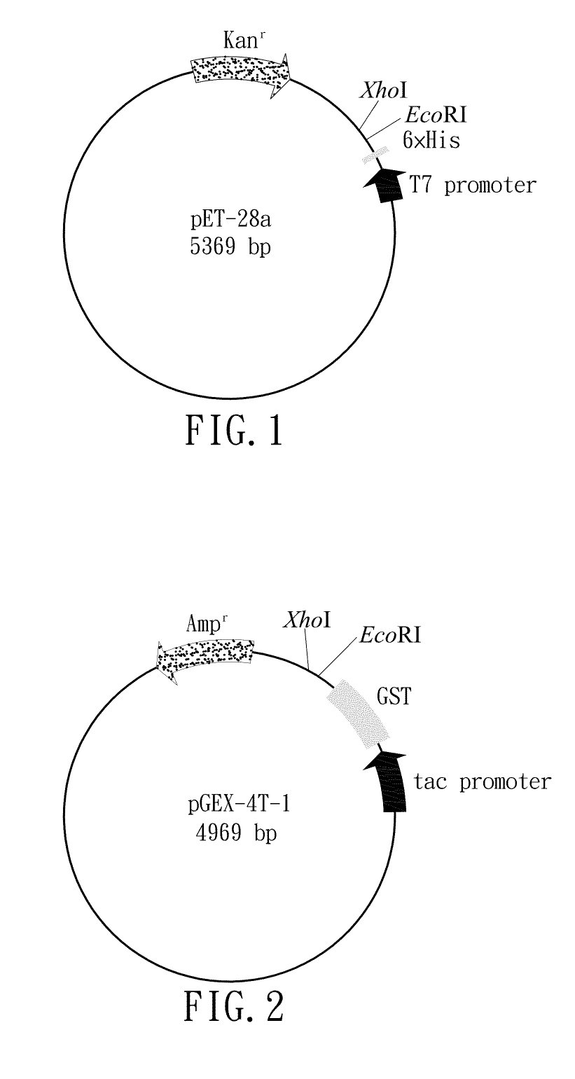

1. The experiments and methods related to DNA cloning as employed in the present invention, such as DNA cleavage reaction by restriction enzymes, agarose gel electrophoresis, polymerase chain reaction (PCR), DNA ligation with T4 DNA ligase, sodium dodecyl sulfate-polyacrylamide gel electrophoresis (SDS-PAGE), Western blotting and plasmid transformation, etc., are referred to a textbook widely known in the art: Sambrook J, Russell D W (2001) Molecular Cloning: a Laboratory Manual, 3rd ed. Cold Spring Harbor Laboratory Press, New York. These techniques can be readily performed by those skilled in the art based on their professional knowledge and experience.
2. Primers used in the Examples were purchased from Genomics BioSci & Tech Co., Ltd.
3. Vectors used in the following Examples are described as follows:
   (1) pET28a vector: A bacterial expression vector, carries a hexahistidine (6×His) tag, T7 promoter, EcoRI and XhoI restriction sites and kanamycin resistance gene (Kan^{r}), 5369 bp (purchased from Novagen, Madison, Wis.) (see FIG. 1).
   (2) pGEX-4T-1 vector: A bacterial expression vector, carries a tac promoter (P_{tac}), glutathione-S-transferase (GST) tag, EcoRI and XhoI restriction sites and ampicillin resistance gene (Amp^{r}), 4969 bp (purchased from GE healthcare, Piscataway, N.J.) (see FIG. 2).
4. Restriction enzymes EcoRI and XhoI, and T4 DNA ligase were purchased from Takara.
5. GSTrap FF affinity column was purchased from GE Healthcare.
6. One Shot® TOP 10F′ competent E. coli cells and BL21 (DE3) competent E. coli cells were purchased from Invitrogen.
7. BL21 (DE3)-pLysS competent E. coli cells were purchased from Stratagene.
8. GeneMark Plasmid Miniprep Plus Purification Kit was purchased from GeneMark.
9. Rabbit anti-chicken IgY conjugated with peroxidase was purchased from Jackson ImmunoResearch Laboratories, Inc. (Cat. No. 303-035-003).
10. ABTS peroxidase substrate was purchased from KPL, Inc.
11. Geneaid Gel Extraction Kit was purchased from Geneaid Biotech Ltd.
12. Transformation:
   The cloned vectors were mixed evenly with 100 μL of competent E. coli cells and placed on ice for 30 minutes. Thereafter, heat-shock of the E. coli cells was carried out at 42° C. for 1.5 minutes in a water bath. The transformation mixture was then placed on ice for 5 minutes. After evenly admixing with 500 μL of LB broth (5 g/L yeast extract, 10 g/L tryptone, 10 g/L NaCl, pH 7.0), the mixture was shake-cultured at 37° C. for one hour. Thereafter, 100 μL of the resultant culture was placed on an agar plate containing kanamycin (50 μg/ml), ampicillin (50 μg/ml) or chloramphenicol (34 μg/ml) and cultured overnight at 37° C. Thereafter, a single antibiotic-resistant colony was selected and inoculated in fresh LB broth containing the same antibiotic as that contained in the agar plate and cultured at 37° C. for 12 to 16 hours.
13. Protein analyses:
   In the following experiments, analyses of protein were performed using sodium dodecyl sulfate polyacrylamide gel electrophoresis (SDS-PAGE) and western blot. The apparatus and chemicals used are described as follows:
   (1) Mini-PROTEAN® Electrophoresis Cell (BioRad) was used for SDS-PAGE analysis.
   (2) Protein transfer was conducted using Mini Trans-Blot® (BioRad) and polyvinylidene difluoride (PVDF) membrane (Millipore).
   (3) Primary and secondary antibodies for fusion protein containing fusion tag in Western blot are shown in Table 1.

TABLE 1

| Fusion tag | Primary antibody | Secondary antibody |
|---|---|---|
| 6 × His | Mouse anti-His antibody | Goat anti-mouse IgG |
| | (Invitrogen, | conjugated with |
| | Cat. No. 37-2900) | alkaline phosphatase |
| GST | Mouse anti-GST antibody | (AP) (Jackson, Cat. No. |
| | (Millipore, | 115-055-003) |
| | Cat. No. 05-782) | |

(4) Chemiluminescence staining was performed using NBT (Biovan, Cat. No. N8100) and BCIP (Biovan, Cat. No. B7500)
14. Genomic DNA of chicken anemia virus (CAV) Taiwanese strain CIA-89, sera from five CAV-infected chickens (i.e., sera 1, 2, 3, 4, and 5) and a serum from a non-infected chicken were kindly provided by Professor Yi-Yang Lien of National Pingtung University of Science and Technology. These sera had been all verified as either being negative or positive for CAV using a commercial ELISA kit purchased from the IDEXX laboratory (CAV Ab Test, catalog number: 99-08702).
15. Determination of CAV ELISA titer values: the CAV ELISA titer values were determined by Chicken Anemia Virus ELISA Kit (purchased from Synbiotics) and are directly proportional to the amount of CAV antibody in the test samples. The titer values were 3447, 5290, 4187, 5567 and 6153 for sera 1, 2, 3, 4, and 5 respectively.

### Example 1

### The Effect of Various Fusion Tags on the Expression on CAV Viral Protein 1 (VP1)

To investigate the effect of fusion tags on the expression of full-length VP1 protein of CAV in E. coli expression systems, wild-type full length vp1 gene was cloned into the pET-28a and pGEX-4T-1 vectors to obtain recombinant plasmids that were used to express hexahistidine (6×His, 1 kDa) and glutathione-s-transferase (GST, approximately 27 kDa) tagged VP1 proteins. These recombinant plasmids were designated as pET-VP1 and pGEX-VP1 respectively. The expression of VP1 protein was induced using isopropyl-β-D-thiogalactopyranoside (IPTG).

### Methods:

### A. Preparing a DNA Fragment Containing Full Length vp1 Gene of Taiwanese Isolate CIA-89

The genomic DNA of CAV was used as a template to obtain a DNA fragment containing the full-length vp1 gene. Forward and reverse primers were designed using the complete coding sequence (NCBI GenBank: U69549.1) of vp1 gene and are designated as F1 and R1, respectively (as shown below). The DNA fragment (1368 bp) containing the full length vp1 gene of Taiwanese isolate CIA-89 was obtained by polymerase chain reaction (PCR) using the conditions listed in Table 2.

```
Forward primer F1 for vp1:
(SEQ ID NO: 1)
5′-gcggaattcatggcaagacgagctcgcaga-3′
            EcoRI
Reverse primer R1 for vp1:
(SEQ ID NO: 2)
5′-gggctcgagtcagggctgcgtcccccagta-3′
              XhoI
```

The primers were designed to contain EcoRI and XhoI restriction sites that are underscored.

TABLE 2

| | Volume |
|---|---|
| PCR Reaction mix | (μL) |
| Genomic DNA of CAV (100 μg/μL) | 1 |
| Forward primer of VP1 F1 (10 mM) | 1 |
| Reverse primer of VP1 R1 (10 mM) | 1 |
| dNTPs (10 mM) | 1 |
| (Ex Taq DNA polymerase buffer) (10X) | 5 |
| Ex Taq DNA polymerase (5 U/μL) | 1 |
| deionized water | 40 |

Thermocycling program: denaturation at 94° C. for 4 minutes. 30 cycles of the followings: denaturation at 94° C. for 1 minutes, primer annealing at 58° C. for 1 minute, extension at 72° C. for 2 minutes. Elongation at 72° C. for 10 minutes.

Obtainment of the PCR product was confirmed by an existence of a 1368 bp band on a 1% agrose gel. The PCR product was purified using Geneaid Gel Extraction Kit according to the manufacturer's instructions. The sequence of the PCR product was identified by Genomics Biosci & Tech and was analyzed using National Center for Biotechnology Information Basic Local Alignment Search Tool (NCBI BLAST). The results showed that the PCR product has a nucleic acid fragment of the vp1 gene represented by SEQ ID NO: 3, which may encode a VP1 protein having an amino acid sequence represented by SEQ ID NO: 11.

B. Constructing pET-VP1 and pGEX-VP1 Recombinant Plasmids

pET-28a and pGEX-4T-1 vectors carrying vp1 gene were designated as pET-VP1 and pGEX-VP1, respectively. Briefly, DNA fragments from pET-28a and pGEX-4T-1 plasmids were incised with EcoRI/XhoI to obtain carrier DNA fragments having 5335 bp and 4954 bp, respectively. In addition, an insert DNA of 1356 bp, which contains full length vp1 gene, was obtained by incising the PCR product obtained in section A with EcoRI/XhoI.

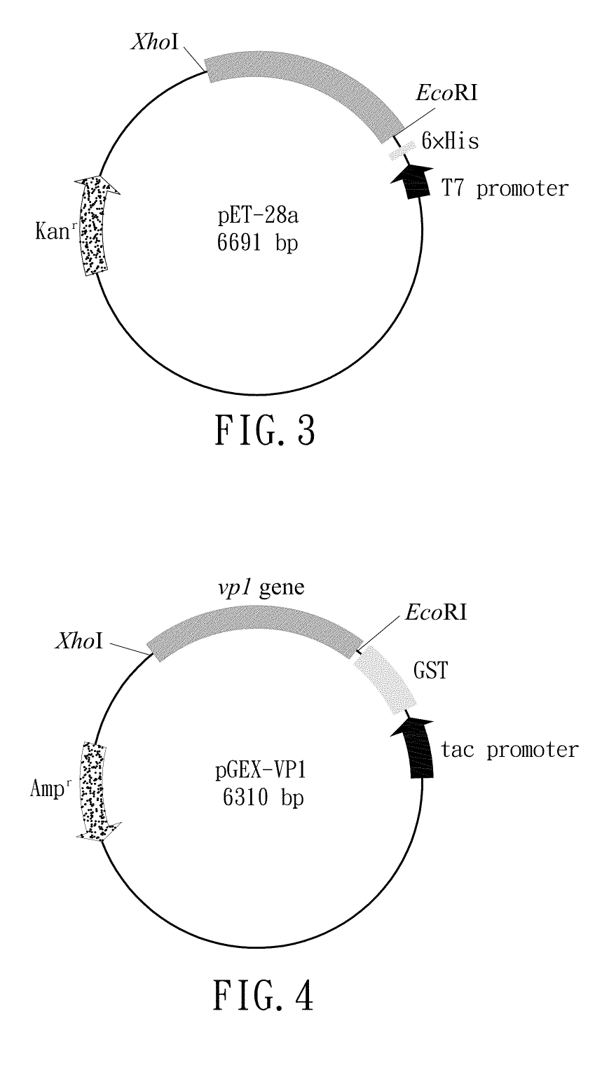

Thereafter, ligation was conducted with a molar ratio of 1:4 of carrier DNA to insert DNA, thereby obtaining pET-VP1 recombinant plasmid (6691 bp, see FIG. 3) and pGEX-VP1 recombinant plasmid (6310 bp, see FIG. 4).

C. Transformation Using E. coli BL21 (DE3) to Express pET-VP1 and pGEX-VP1

Each of the recombinant plasmids thus obtained was transformed into One Shot® TOP 10F′ competent E. coli following the procedures as set forth in <Materials and methods>section item 12, Transformation, followed by cultivation in the presence of kanamycin and ampicillin. The recombinant plasmids were purified using GeneMark Plasmid Miniprep Plus Purification Kit according to the manufacturer's instructions. The sequences for the recombinant plasmids were confirmed by Genomics BioSci & Tech Co., Ltd.

The pET-VP1 and pGEX-VP1 thus confirmed were further separately transformed into competent E. coli BL21 (DE3) based on the procedure set forth in <Materials and methods>section item 12, Transformation. Two negative controls were obtained by separately transforming competent E. coli BL21 (DE3) with pET-28a and pGEX-4T-1 plasmids.

D. Induction of Tagged-VP1 Fusion Protein in E. coli BL21 (DE3) Using isopropyl-β-D-thiogalactopyranoside (IPTG)

E. coli. BL21 (DE3) transformed with pET-VP1 and E. coli BL21 (DE3) transformed with pET-28a (negative control) were inoculated separately in LB broth containing 50 μg/mL of kanamycin and cultured overnight at 37° C. to obtain precultures. Similarly, E. coli BL21 (DE3) transformed with pGEX-VP1 and E. coli BL21 (DE3) transformed with pGEX-4T-1 (negative control) were inoculated in LB broth containing 50 μg/mL of ampicillin and cultured overnight at 37° C. to obtain precultures. The precultures were diluted 100-fold with fresh LB broth containing 50 μg/mL of the respective antibiotics and further cultured at 37° C. in fresh LB broth containing the respective antibiotics until an optical density (OD₆₀₀) of each of the cultures had reached 0.4 to 0.5. Each of the cell cultures was added with a final concentration of 1 mM IPTG and cultured at 37° C. for 4 hours to induce the transformed E. coli BL21 (DE3) to express a tagged-VP1 fusion protein. 1 ml of each of the cell cultures was collected before and after 4 hours of IPTG induction to determine the expression of the tagged-VP1 fusion protein. Specifically, a whole cell lysate was prepared by obtaining a cell pellet by centrifugation for 10 min at 10,000 rpm and resuspended in 1× sample loading buffer, which was obtained by diluting 4× sample loading buffer (0.15 M Tris-HCl, 4.5% SDS, 20% β-mercaptoethanol and 0.1% bromophenol blue) with PBST (1.44 g/LKH₂PO₄, 9.0 g/L NaCl, 0.795 g/L Na₂HPO₄ and 0.1% Tween 20). This was followed by protein denaturation on heat block at 100° C. for 10 minutes and centrifuged at 10,000 rpm for 1 minute to collect the protein in the supernantant.

Protein expression levels were determined by SDS-PAGE and Western blot as set forth in <Material and method>item 13, Protein analyses.

### Results:

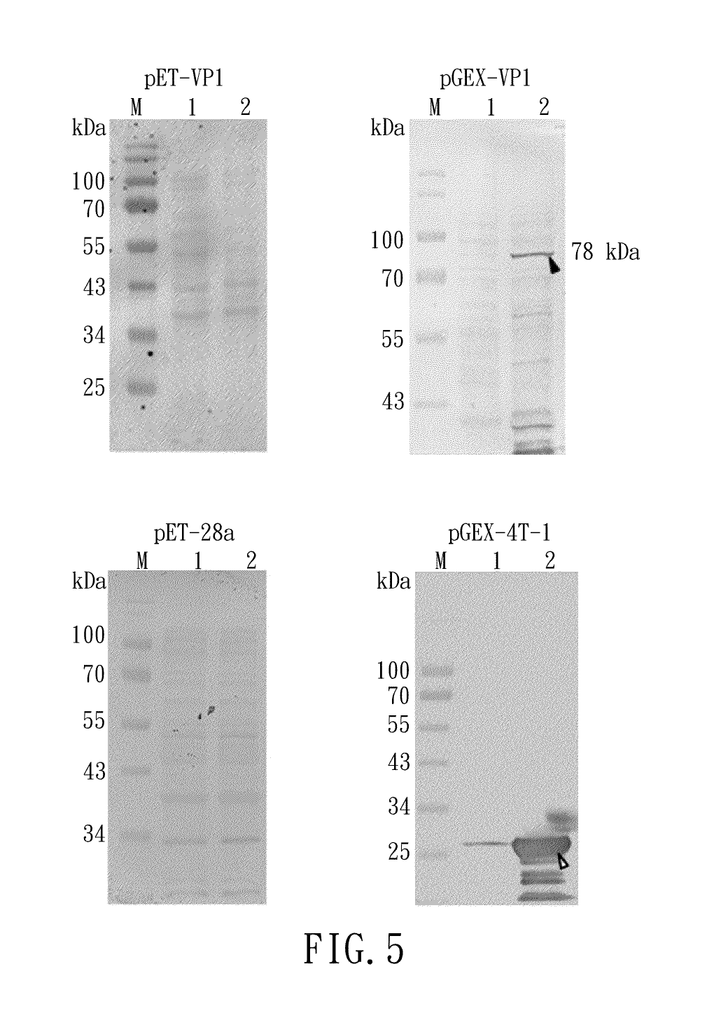

FIG. 5 illustrates Western blots showing expression of tagged-VP1 fusion protein from E. coli BL21 (DE3) transformed with either pET-VP1 or pGEX-VP1 before and after IPTG induction. M indicates protein ladder marker. Lanes 1 and 2 indicate expression of tagged-VP1 fusion proteins from E. coli BL21 (DE3) before and after IPTG induction, respectively.

As shown in FIG. 5, the full length GST-tagged VP1 protein detected by monoclonal anti-GST antibody was expressed to an obvious extent in E. coli BL21 (DE3) harboring pGEX-VP1 after IPTG induction for 4 hours (lane 2 of the upper right panel of FIG. 5, solid triangle), while the protein samples containing pGEX-VP1 plasmid without IPTG induction did not show a band at 78 kDa (lane 1 of upper right panel of FIG. 5). In contrast, 6×His-VP1 protein (52 kDa) was almost undetectable by Western blot when anti-His monoclonal antibody was used (lane 2 of the upper left panel of FIG. 5). Negative controls using pET-28a and pGEX-4T-1 plasmids showed a fusion tag (hollow triangle in the lower right panel indicates GST) with no expression of the VP1 protein (lower left and right panels).

These results illustrate that the GST fusion tag improved full-length CAV VP1 protein expression significantly in E. coli BL21 (DE3), which might suggest that the GST fusion tag promotes correct folding of the GST-VP1 fusion protein to improve the stability of the fusion protein. Thus, as compared to the 6×His-VP1 protein, the GST-VP1 fusion protein is unlikely to be degraded.

### Example 2

### Rare Codon Analysis and Optimization of vp1 Gene

Rare codons of vp1 gene (SEQ ID NO: 3) of Taiwan isolate CIA-89 were identified using GeneScript rare codon analysis tool software. From the analysis, 14% of the rare codons exist in the full length vp1 gene. Clusters of rare codon exist from base pairs 1 to 90, 1 to 180 and 1 to 321 and represent 46%, 41% and 26% of the codons, respectively. Clusters of rare codons present in the 5′ end of vp1 gene may affect the expression of VP1 protein in E. coli.

In order to enhance the expression level of VP1 protein, rare codons in the 5′ end of the vp1 gene (i.e., a coding region of the vp1 gene) were optimized to E. coli's preferred codons from base pairs 1-321 starting from the 5′ end of the vp1 gene. The information for codon replacements was obtained according to Genscript OptimumGene™ software. The codon substitution was performed without altering the amino acid sequence that will be expressed in E. coli. The codon optimized fragment is hereinafter referred to as “5′-opt-vp1” fragment. The 321 bp DNA fragment from 5′ end of the vp1 gene was subjected to codon optimization. The 321 bp DNA fragment that corresponds to 5′ end of the vp1 gene was subjected to rare codon optimization as follows: AGA/AGG/CGA/CGC/CGG to CGT (R), CCC to CCG (P), CTC/CTT/TTG to CTG (L), GGA/GGC/GGG to GGT (G), ACA/ACT/ACG to ACC (T), CAA to CAG (Q) and ATA/ATT to ATC (I)). The codon optimization further includes the following codon replacements: GCA/GCC/GCG to GCT (A), AAG to CGT (K), CAT to CAC (H), TTT to TTC (F), AGC/AGT/TCC to TCT (S), TAT to TAC (Y) and GTC/GTG to GTT (V). The codons replacements of the wild-type CAV vp1 gene from Taiwanese isolate CIA-89 is shown in Table 3. The optimized fragment was synthesized by Genomics Biosci & Tech Co. and has a sequence of SEQ ID NO: 4.

TABLE 3

| Codon of vp1 | | |
|---|---|---|
| gene of | | Corresponding |
| Taiwanese | Optimized | amino |
| isolate CIA-89 | codon | acid |
| gca | gct | Alanine |
| gcc | | |
| gcg | | |
| aga | cgt | Arginine |
| agg | | |
| cga | | |
| cgc | | |
| cgg | | |
| caa | cag | Glutamine |
| gga | ggt | Glycine |
| ggc | | |
| ggg | | |
| cat | cac | Histidine |
| ata | atc | Isoleucine |
| att | | |
| ctc | ctg | leucine |
| ctt | | |
| ttg | | |
| aag | aaa | Lysine |
| ttt | ttc | Phenylalanine |
| ccc | ccg | Proline |
| cct | | |
| agc | tct | Serine |
| agt | | |
| tcc | | |
| aca | acc | Threonine |
| act | | |
| acg | | |
| tat | tac | Tyrosine |
| gtc | gtt | Valine |
| gtg | | |

### Example 3

### Optimization of Codon in the 5′ End of vp1 Gene Enhances the Expression of Recombinant CAV vp1 Protein in E. coli

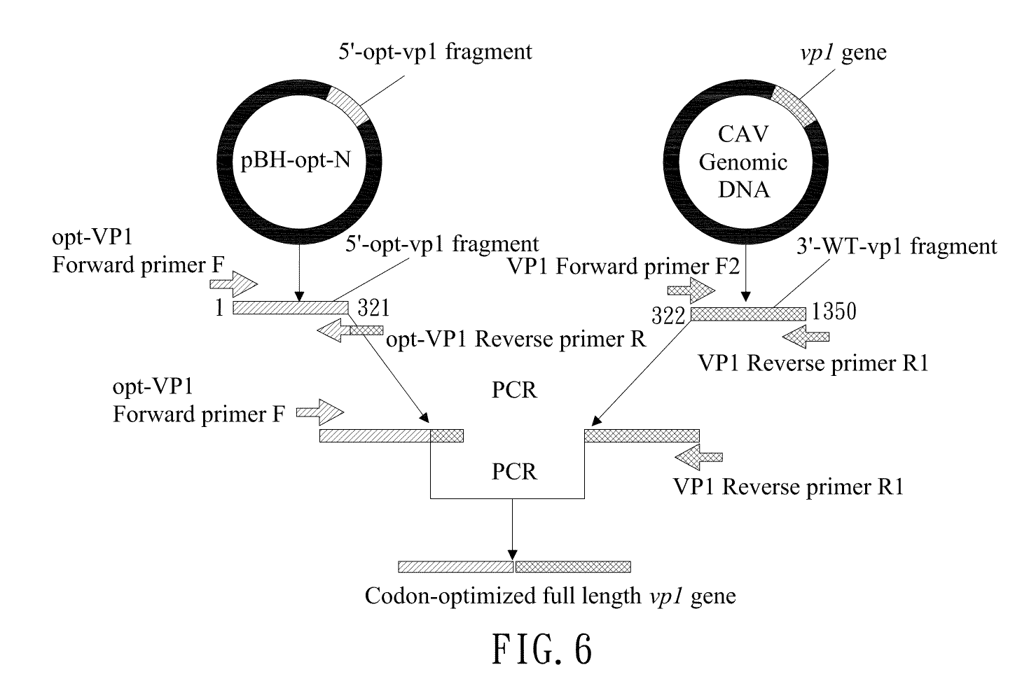

The 5′-opt-vp1 fragment, (base pairs 1 to 321, SEQ ID NO: 4) was fused with the 3′ end of the wild-type vp1 gene (base pairs 322 to 1350, hereinafter referred to as “3′-WT-vp1” fragment) to give an intact open reading frame, thus to assess the effect of rare codon optimization at the 5′ end on VP1 protein expression. Construction of a full length vp1 DNA fragment containing the 5′-opt-vp1 fragment fused with the 3′-wild-type-vp1 fragment was conducted using an overlapping PCR strategy as shown in a schematic flow chart of FIG. 6. The primers used for the overlapping PCR strategy are listed in Table 4.

TABLE 4

| primer | Nucleotide sequence (5′→3′) |
|---|---|
| opt-VP1 | EcoRI |
| Forward | cccgaattcatggctcgtcgtgctcgtcgt |
| primer F | (SEQ ID NO: 6) |
| opt-VP1 | cgctagcaggaactctttcaggttaacagagattttagcaacacg |
| Reverse | agc |
| primer R | (SEQ ID NO: 7) |
| VP1 Forward | aacctgaaagagttcctgctagcg |
| primer F1 | (SEQ ID NO: 8) |
| VP1 Reverse | XhoI |
| primer R1 | gggctcgagtcagggctgcgtccoccagta |
| | (SEQ ID NO: 2) |

Note:
underlined nucleotides are the cutting sites for the corresponding restriction enzymes.

### Methods:

A. Preparation of the Full Length vp1 Gene Containing 5′-opt-vp1 Fragment with 3′-WT-vp1 gene

The 5′-opt-vp1 (SEQ ID NO: 4) obtained in Example 2 was cloned into a pBH vector to obtain a recombinant vector, pBH-opt-N (3255 bp) (SEQ ID NO: 5). This recombinant vector contains an ampicillin resistant gene and the 5′-opt-vp1 fragment and was used as a template in the PCR reaction having conditions listed in Table 5 to obtain a 354 bp PCR product having 5′-opt-vp1 fragment.

TABLE 5

| | Volume |
|---|---|
| PCR reaction mix | (μL) |
| Recombinant plasmid pBH-co-N (50 μg/μL) | 1 |
| opt-VP1 Forward primer F (10 mM) | 1 |
| opt-VP1 Reverse primer R (10 mM) | 1 |
| dNTPs (10 mM) | 1 |
| Ex Taq DNA polymerase buffer (10X) | 5 |
| Ex Taq DNA polymerase (5 U/μL) | 1 |
| Deionized water | 40 |

Thermocycling program: denaturation at 94° C. for 4 minutes. 30 cycles of the following: denaturation at 94° C. for 1 minute, primer annealing at 58° C. for 1 minute, extension at 72° C. for 2 minutes. Elongation at 72° C. for 10 minutes.

The PCR product was confirmed by existence of a 354 bp band on a 1% agrose gel, and was purified using Geneaid Gel Extraction Kit.

In order to obtain the 3′-WT-vp1 fragment (SEQ ID NO: 9), genomic DNA of CAV vp1 gene was used as a template. VP1 Forward primer F2 and VP1 Reverse primer R1 were used in the PCR having conditions listed in Table 6.

TABLE 6

| | Volume |
|---|---|
| PCR reaction mix | (μL) |
| CAV genomic DNA (50 μg/μL) | 1 |
| VP1 Forward primer F2 (10 mM) | 1 |
| VP1 Reverse primer R1 (10 mM) | 1 |
| dNTPs (10 mM) | 1 |
| Ex Taq DNA polymerase buffer (10X) | 5 |
| Ex Taq DNA polymerase (5 U/μL) | 1 |
| Deionized water | 40 |

Thermocycling program: denaturation at 94° C. for 4 minutes. 30 cycles of the following: denaturation at 94° C. for 1 minute, primer annealing at 58° C. for 1 minute, extension at 72° C. for 2 minutes. Elongation at 72° C. for 10 minutes.

The PCR product containing the 3′-WT-vp1 fragment was confirmed by an existence of a 1038 bp band on a 1% agrose gel, and was purified using Geneaid Gel Extraction Kit.

The full length vp1 gene containing the 5′-opt-vp1 fragment and 3′-WT-vp1 was obtained by fusing the two fragments together using overlapping PCR strategy.

Specifically, the purified PCR products of 5′-opt-vp1 and 3′-WT-vp1 fragments were dissolved in deionized water and used as templates in the PCR reaction having conditions as listed in Table 7. The PCR product is designed to contain an EcoRI restriction site upstream of the 5′ end of the VP1 gene (at the position of base pairs 4 to 9, gaattc), and an XhoI restriction site downstream of the 3′ end of the VP1 DNA (at the position of base pairs 1360 to 1365, ctcgag).

TABLE 7

| | Volume |
|---|---|
| PCR reaction mix | (μL) |
| PCR product containing the opt | 3 |
| fragment (19.6 μg/μL) | |
| PCR product containing the wild-type | 2.8 |
| fragment (62.5 μg/μL) | |
| co-VP1 Forward primer F (10 mM) | 1 |
| VP1 Reverse primer R1 (10 mM) | 1 |
| dNTPs (10 mM) | 1 |
| Ex Taq DNA polymerase buffer (10X) | 5 |
| Ex Taq DNA polymerase (5 U/μL) | 1 |
| Deionized water | 35.2 |

Thermocycling program: denaturation at 94° C. for 3 minutes. 30 cycles of the following: denaturation at 94° C. for 45 seconds, primer annealing at 58° C. for 45 seconds, extension at 72° C. for 1 minute. Elongation at 72° C. for 10 minutes.

The PCR product including the full length vp1 gene that contains the 5′-opt-vp1 fragment and 3′-WT-vp1 fragment was confirmed by an existence of a 1368 bp band on a 1% agrose gel. The PCR product was purified using Geneaid Gel Extraction Kit.

The sequence of the opt-vp1 fragment from the purified PCR product was identified by Genomics Biosci & Tech. and analyzed by NCBI BLAST. The result indicates that the opt-vp1 fragment contains the full length vp1 gene that includes the 5′-opt-vp1 fragment and 3′-WT-vp1 fragment, and that is represented by SEQ ID NO: 10.

B. Construction of a pGEX-opt-VP1 Recombinant Plasmid Containing the opt-vp1 Fragment

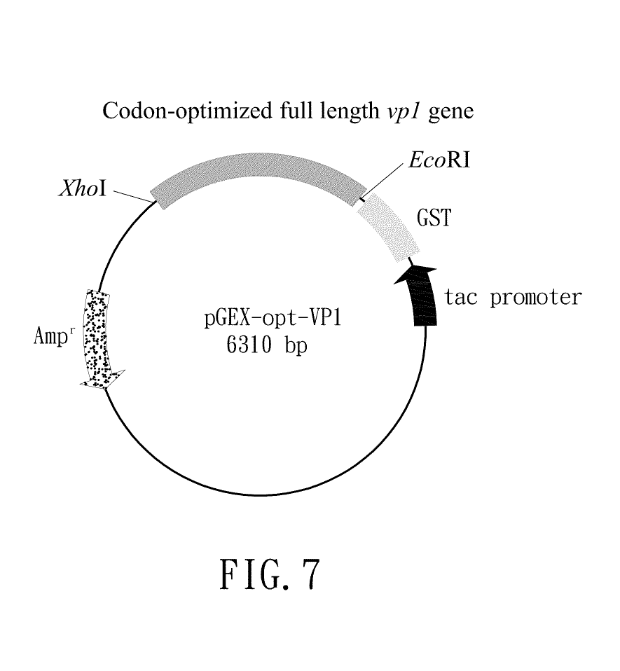

EcoRI/XhoI were used to incise a pGEX-4T-1 plasmid to obtain a 4954 bp carrier DNA. In addition, EcoRI/XhoI were used to incise the opt-vp1 fragment (1368 bp) of section A, thus obtaining a 1356 bp insert DNA that contains the 5′ codon-optimized full length vp1 gene and hereinafter referred to as opt-vp1 fragment. Thereafter, ligation was conducted with a molar ratio of 1:4 of carrier DNA to insert DNA to obtain a pGEX-opt-VP1 recombinant plasmid (6310 bp, see FIG. 7).

pGEX-VP1 and pGEX-opt-VP1 plasmids were separately transformed into One Shot® TOP 10F′ E. coli competent cells based on the procedures set forth in <Materials and methods>item 12. The transformed cells were cultured in the presence of ampicillin and collected by centrifugation. The amplified plasmids were obtained using GeneMark Plasmid Miniprep Plus Purification Kit. The sequence of the purified plasmids were identified and confirmed by Genomics Biosci & Tech.

C. E. coli Strain BL21 (DE3)-pLysS Transformed with pGEX-opt-VP1 and pGEX-VP1

pGEX-VP1 and pGEX-opt-VP1 plasmids obtained in the above section B were further transformed into competent E. coli BL21 (DE3)-pLysS strain based on the procedure as set forth in <Materials and methods>item 12, Transformation, so as to obtain two transformants, i.e., E. coli. BL21 (DE3)-pLysS strain containing pGEX-opt-VP1 and E. coli. pGEX-VP1 recombinant plasmids.

D. IPTG Induction of E. coli BL21 (DE3)-pLysS to Express Full Length Wild-Type VP1 Protein and Optimized VP1 Protein

Each of the transformants obtained from the aforementioned item C. was inoculated in LB broth containing 50 μg/mL of ampicillin and 50 μg/mL of chloramphenicol, and cultured at 37° C. overnight to obtain a preculture. The preculture was diluted 100-fold with a fresh LB broth containing 50 μg/mL of ampicillin and 50 μg/mL of chloramphenicol and further cultured at 37° C. until an optical density (OD₆₀₀) of the cultures had reached 0.4 to 0.5. The culture was added with a final concentration of 1 mM IPTG and cultured at 37° C. for 4 hours to induce the transformants to express GST-VP1 and GST-opt-VP1 fusion proteins, respectively.

1 mL of the culture was obtained before and after IPTG induction, i.e., pre-induction (0 hours) and post-induction at 1, 2, 3, and 4 hours later. The protein samples were obtained from the whole cell lysate by centrifuging at 10,000 rpm at 4° C. for 10 minutes and resuspending in lysis buffer. Further preparation of the protein samples was similar to that as described in Example 1, section D.

Protein expression levels were determined by SDS-PAGE and Western blot as set forth in <Material and method>item 13, Protein analyses. The quantity of the protein was determined by AlphaDigi™ (purchased from Unimed Healthcare Inc.). A standard curve was established by measuring known concentrations of GST-opt-VP1. The concentration of the fusion proteins GST-VP1 (nmol) and GST-opt-VP1 (nmol) were determined by extrapolating the signal intensity of the protein samples to the standard curve and further converted into units of mg/mL.

### Results:

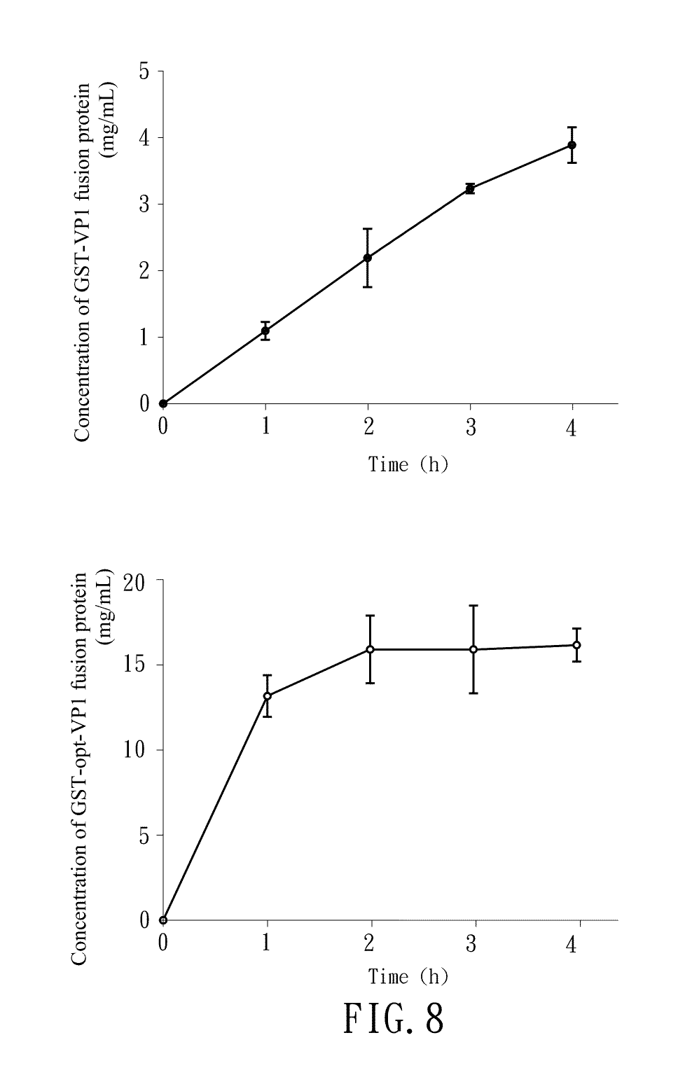

FIG. 8 illustrates plots showing the protein levels of GST-VP1 and GST-opt-VP1 in E. coli strain BL21 (DE3)-pLysS before and after IPTG induction.

As shown in FIG. 8, the quantitative yield for the wild-type VP1 protein expression is 3.87 mg/mL, whereas the GST-opt-VP1 protein in BL21 (DE3)-pLysS reached 17.5 mg/mL, representing a 4.6 fold increase in the optimized protein after four hours of IPTG induction. These results indicate that the optimization of the codons located at 1 to 321 bp can highly increase the VP1 protein expression in E. coli. strain BL21 (DE3)-pLysS.

### Example 4

### Antigenicity of the Recombinant GST-opt-VP1 Protein

### A. Purification of Recombinant VP1 Protein Using GST Affinity Chromatography

The GST-opt-VP1 protein in E. coli. BL21 (DE3)-pLysS was purified using GSTrap FF affinity column (GE healthcare, Piscataway, N.J.). Briefly, to purify the recombinant VP1 protein, after 1 hour of induction with IPTG, the cells were spun down from 50 mL of culture and resuspended in GST resin binding buffer (140 mM NaCl, 2.7 mM KCl, 10 mM Na₂HPO₄, 1.8 mM KH₂PO₄, pH 7.3), followed by sonication on ice three times for 3 minutes with a 20% pulsed activity cycle (MISONIX Sonicator® 3000). The lysate was then centrifuged for 10 min at 10,000 rpm to remove the cell debris. The resulting cell supernatant was applied to a GSTrap FF affinity column packed with 1 mL of GSTrap resin at a flow rate of 0.5 ml/min. 10-fold volume of the binding buffer was used to wash out the unbound protein, and then the bound proteins were eluted with an eluent (50 mM Tris-HCl, 10 mM reduced glutathione, pH 8.0) with a flow rate of 1 ml/min. Each fraction consisted of 1 ml of elutant. The fractions were monitored at OD₂₈₀ using the optical unit of a liquid chromatography system (AKTAprime plus, GE Healthcare BioScience AB, Uppsala, Sweden). The fractions showing an optical peak for the recombinant CAV VP1 protein were collected for analysis. The VP1 protein concentration was determined using a Micro BCA kit (Pierce, Rockford, Ill.) using bovine serum albumin as the reference protein.

To confirm the identity of the recombinant GST-opt-VP1 protein purified by GSTrap FF column, the proteins were separated by 12.5% SDS-PAGE. The relevant band was then excised from the 12.5% SDS-PAGE gel after Coomassie blue staining and was digested with trypsin. The resulting samples were subjected to the MALDI-TOF-MS mass spectrometry (ESI-QUAD-TOF) (Biotechnology Center, National Chung Hsing University) to allow amino acid sequence identification as described in a previous study disclosed by Lee et al. (Lee M. S. et al. (2009), Process Biochem., 44:390-395).

In addition, GST protein was prepared and used for background measurements in the following indirect enzyme linked immunosorbent assay (ELISA). Briefly, GST protein was obtained by transforming pGEX-4T-1 in E. coli. BL21 (DE3)-pLysS and cultured with ampicillin and chloramphenicol as set forth in <Materials and methods>item 13, Transformation. The GST protein in the transformed E. coli BL21 (DE3)-pLysS was also purified and identified using the same procedures as those used for GST-opt-VP1 protein.

### B. Enzyme Linked Immunosorbent Assay (ELISA)

The antigenicity of the purified E. coli-expressed GST-opt-VP1 protein was evaluated by ELISA assay, in which E. coli-expressed GST-opt-VP1 protein was used as a coating antigen. Briefly, 100 μL of coating buffer (0.15 mM Na₂CO₃, 0.35 mM NaHCO₃ and 0.03 M NaN₃, pH 9.5) containing 2 μg of the purified recombinant GST-opt-VP1 protein from the above section A was added in each well of a 96-well plate, and allowed to stand at 4° C. for 16 hours. Thereafter, the supernatant was removed from each of the wells and 200 μL of 5% skim milk was added into each well, followed by incubation at 37° C. for 1.5 hours. The supernatant in each well was removed and the well was washed with 0.1% Tween 20 (in PBS, i.e. PBST) three times. Sera 1 to 6 obtained from <Materials and methods>item 14 which were diluted 200 fold with PBST were added to the wells and allowed to react at 37° C. for 2 hours. The supernatant was removed and the wells were washed with a washing buffer (PBST) for five times. 200 μL of anti-chicken rabbit IgY conjugated with peroxidase (diluted 5000 fold with PBST) was added to each well and allowed to react under 37° C. for 1.5 hours. Thereafter, the supernatant was removed and the wells were washed with the washing buffer (PBST) three times. 100 μL of ABTS peroxidase substrate was added to each well, followed by 20 minutes of incubation for color development. Lastly, the absorbance for each well at 405 nm (OD₄₀₅) was determined using an ELISA reader (Dynex Technologies, USA). In addition, the OD₄₀₅ value of the GST protein obtained from the above section A was used as a background value. A cut-off value was defined as the mean of the OD₄₀₅ value of CAV-negative sera±2 fold standard deviation.

### Results:

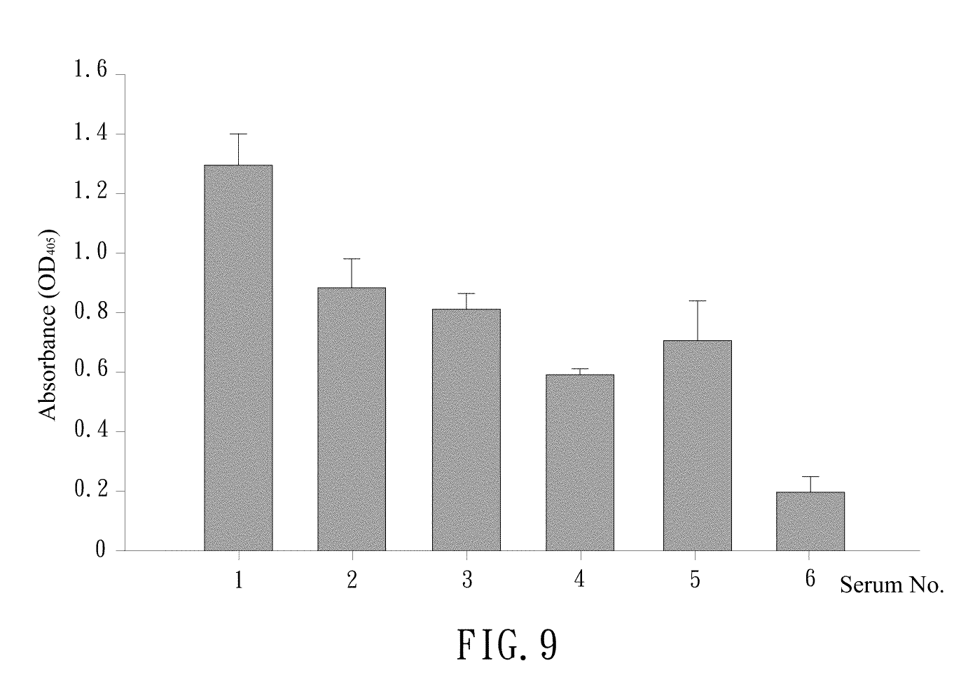

As shown in FIG. 9, the purified GST-opt-VP1 protein showed higher antigenicity with the infected chicken sera (CAV-positive sera, sera No. 1 to 5) than with the non-infected chicken serum 6 (CAV-negative serum)since the OD₄₀₅ values of all CAV-positive sera were higher than the cut-off value. The OD₄₀₅ values for the infected chicken sera were 1.29, 0.88, 0.81, 0.59 and 0.71 for sera nos. 1 to 5, respectively.

These data indicate that CAV positive/negative sera can be successfully discriminated using the purified GST-opt-VP1 protein. Thus, the N-terminus optimized full length VP1 protein shows considerable antigenic potential and is able to pinpoint sera from chickens that are infected with CAV.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

What is claimed is:

1. An expression cassette adapted to be expressed in an E. coli host cell and comprising a first nucleic acid fragment encoding a full-length chicken anemia virus (CAV) VP1 protein,
wherein the first nucleic acid fragment has a 5′-region that encodes a N-terminal amino acid sequence of the full-length CAV VP1 protein and is codon-optimized as compared to a corresponding 5′-region of a wild-type CAV vp1 gene encoding the full-length VP1 protein; and
wherein the 5′-region of the first nucleic acid fragment contains therein optimized codons that are introduced into the corresponding 5′-region of the wild-type CAV vp1 gene by codon optimization that includes the following rare codon replacements:
replacing a glycine codon of gga, ggc or ggg with a codon of ggt;
replacing a leucine codon of ctc, ctt or ttg with a codon of ctg;
replacing a threonine codon of aca, act or acg with a codon of acc;
replacing an isoleucine codon of ata or att with a codon of atc;
replacing a glutamine codon of caa with a codon of cag;
replacing an arginine codon of aga, agg, cga, cgc or cgg with a codon of cgt; and
replacing a praline codon of ccc or cct with a codon of ccg.

2. The expression cassette according to claim 1, wherein the codon optimization further includes the following codon replacements:
replacing an alanine codon of gca, gcc or gcg with a codon of gct;
replacing a lysine codon of aag with a codon of cgt;
replacing a histidine codon of cat with a codon of cac;
replacing a phenylalanine codon of ttt with a codon of ttc;
replacing a serine codon of agc, agt or tcc with a codon of tct;
replacing a tyrosine codon of tat with a codon of tac; and
replacing a valine codon of gtc or gtg with a codon of gtt.

3. The expression cassette according to claim 1, wherein the N-terminal amino acid sequence of the full-length CAV VP1 protein encoded by the 5′-region of the first nucleic acid fragment has a length of at least 30 to 130 amino acids.

4. The expression cassette according to claim 1, wherein the N-terminal amino acid sequence of the full-length CAV VP1 protein encoded by the 5′-region of the first nucleic acid fragment has a length of 107 amino acids.

5. The expression cassette according to claim 1, wherein the 5′-region of the first nucleic acid fragment encodes at least 30 contiguous amino acids as calculated from the N-terminal of the full-length CAV VP1 protein.

6. The expression cassette according to claim 1, wherein the full-length CAV VP1 protein has an amino acid sequence of SEQ ID NO: 12.

7. The expression cassette according to claim 1, wherein the full-length CAV VP1 protein has an amino acid sequence of SEQ ID NO: 11.

8. The expression cassette according to claim 1, wherein the first nucleic acid fragment has a nucleotide sequence of SEQ ID NO: 10.

9. The expression cassette according to claim 1, wherein the expression cassette further comprises a second nucleic acid fragment operably connected to the first nucleic acid fragment and encoding a target protein.

10. The expression cassette according to claim 9, wherein the second nucleic acid fragment is located upstream of the first nucleic acid fragment, so that the first and second nucleic acid fragments together encode a fusion protein of said full-length CAV VP1 protein and said target protein, wherein said target protein is located upstream of the N-terminal acid sequence of the full-length CAV VP1 protein.

11. The expression cassette according to claim 9, wherein the target protein is a protein tag, an antibody, an antigen, an antimicrobial peptide, a hormone peptide or an enzyme.

12. The expression cassette according to claim 11, wherein the target protein is a protein tag selected from the group consisting of glutathione-S-transferase, hexahistidine tag, maltose binding protein, small ubiquitin-like modifier, and combinations thereof.

13. The expression cassette according to claim 12, wherein the target protein is glutathione-S-transferase.

14. The expression cassette according to claim 1, wherein the expression cassette further comprise a promoter that operably controls the expression of the first nucleic acid fragment.

15. The expression cassette according to claim 1, wherein the expression cassette is in a form of a plasmid or vector expressible in the E. coli host cell.

16. A recombinant plasmid or vector expressible in an E. coli host cell, wherein recombinant plasmid or vector carries the expression cassette of claim 1.

17. A recombinant E. coli cell carrying the expression cassette of claim 1.
